(19) United States
(12) Patent Application Publication
(10) Pub. No.: US 2011/0270138 A1
(43) Pub. Date: Nov. 3, 2011

(54) ULTRASOUND MACRO-PULSE AND MICRO-PULSE SHAPES FOR NEUROMODULATION

(76) Inventor: David J. Mishelevich, Playa del Rey, CA (US)

(21) Appl. No.: 13/098,473

(22) Filed: May 1, 2011

Related U.S. Application Data
(60) Provisional application No. 61/330,363, filed on May 2, 2010.

(51) Int. Cl. A61N 7/00

(52) U.S. Cl. 601/2

(57) ABSTRACT

Disclosed are methods and systems for non-invasive ultrasound stimulation of neural structures, whether the central nervous systems (such as the brain), nerve roots, or peripheral nerves using macro- and micro-pulse shaping. Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results.

### CROSS REFERENCE TO RELATED APPLICATIONS

This provisional patent application does not claim priority to any other patent application.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for ultrasound neuromodulation of the brain and other neural structures.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. One or a plurality of neural elements can be neuromodulated.

Potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels which resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

While the ultrasonic frequencies for neural stimulation are known, it would be preferable to use macro- and micro-pulse shapes optimized for neuromodulation.

### SUMMARY OF THE INVENTION

It is the purpose of this invention to provide methods and systems and methods for optimizing the macro- and micro-pulse shapes used for ultrasound neuromodulation of the brain and other neural structures. Ultrasound neuromodulation is accomplished superimposing pulse trains on the base ultrasound carrier. For example, pulses spaced at 1 Hz of 250 μsec in duration may be superimposed on an ultrasound carrier of 500 kHz. Macro-pulse shaping refers to the overall shaping of the individual pulses delivered at so many Hz (e.g., the pulses appearing at 1 Hz). Micro-pulse shaping refers to the shaping of the individual constituent waveforms in the carrier (e.g., 500 kHz). Either the macro-pulse shapes or the micro-pulse shapes can be sine waves, square waves, triangular waves, or arbitrarily shaped waves. Neither needs to consistent, that is all being the same shape (e.g., all sine waves); heterogeneous mixtures are permitted (e.g., sine waves mixed with square waves) either within the macro or micro or between the macro and micro. Functional output and/or Positron Emission Tomography (PET) or fMRI imaging can judge the results. In addition, the effect on a readily observable function such as stimulation of the palm and assessing the impact on finger movements can be done and the effect of changing of the macro-pulse and/or micro-pulse characteristics observed.

### BRIEF DESCRIPTION OF THE DRAWINGS

FIG. 1 shows a diagram of macro-pulse shaping.

FIG. 2 shows a diagram of micro-pulse shaping.

FIG. 3 shows a block diagram of the system for generating the output incorporating macro- and micro-pulse shaping.

### DETAILED DESCRIPTION OF THE INVENTION

It is the purpose of this invention to provide methods and systems and methods for non-invasive ultrasound stimulation of neural structures, whether the central nervous systems (such as the brain), nerve roots, or peripheral nerves using macro- and micro-pulse shaping. Ultrasound neuromodulation can be used to treat a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. It can be also applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected. In addition, the effect on a readily observable function such as stimulation of the palm and assessing the impact on finger movements can be done and the effect of changing of the macro-pulse and/or micro-pulse characteristics observed.

The acoustic frequency (e.g., typically in the range of 0.3 MHz to 0.8 MHz or above whether cranial bone is to be penetrated or not) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). Power is generally applied at a level less than 60 mW/cm2. Ultrasound pulses may be monophasic or biphasic, the choice made based on the specific patient and condition. Ultrasound stimulators are well known and widely available.

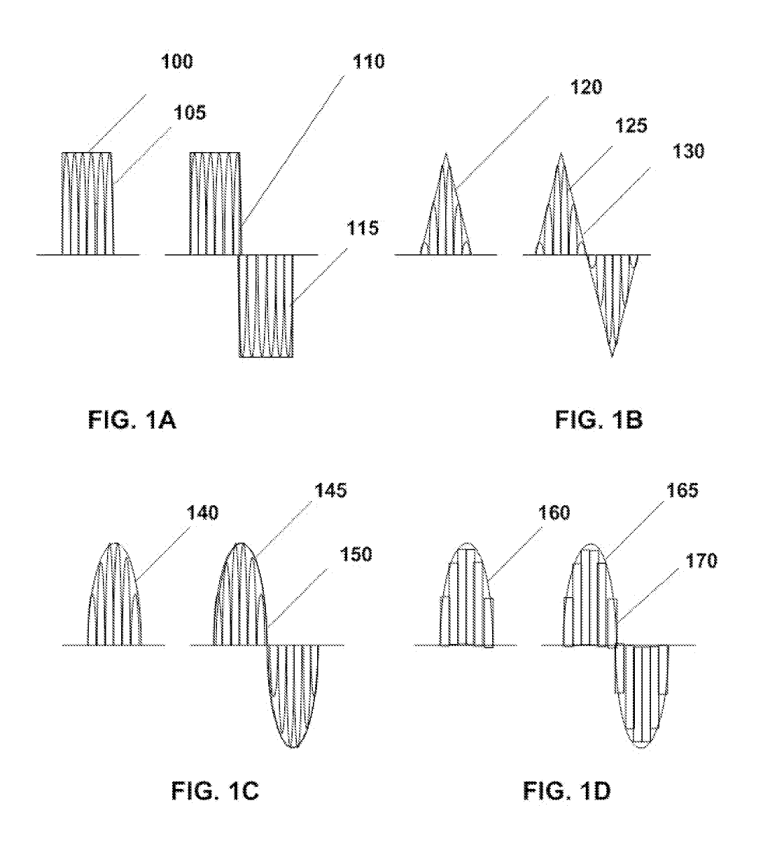

FIG. 1 demonstrates macro-pulse shaping defined as the overall shape of the pulse burst. The individual pulses making up the macro-pulse shapes are the micro-pulse shapes. FIG. 1A shows monophasic square-wave macro-pulse 100 and biphasic square-wave macro-pulse 110 made up of sine-wave micro-pulses 105. FIG. 1B illustrates monophasic triangular macro-pulse 120 and biphasic triangular macro-pulse 130 made up of sine-wave micro-pulses 125. FIG. 1C illustrates monophasic sinusoidal macro-pulse 140 and biphasic sinusoidal macro-pulse 150 made up of sine-wave micro-pulses 145. FIG. 1D illustrates monophasic sinusoidal macro-pulse 160 and biphasic sinusoidal macro-pulse 170, in this case made up of square-wave micro-pulses 165.

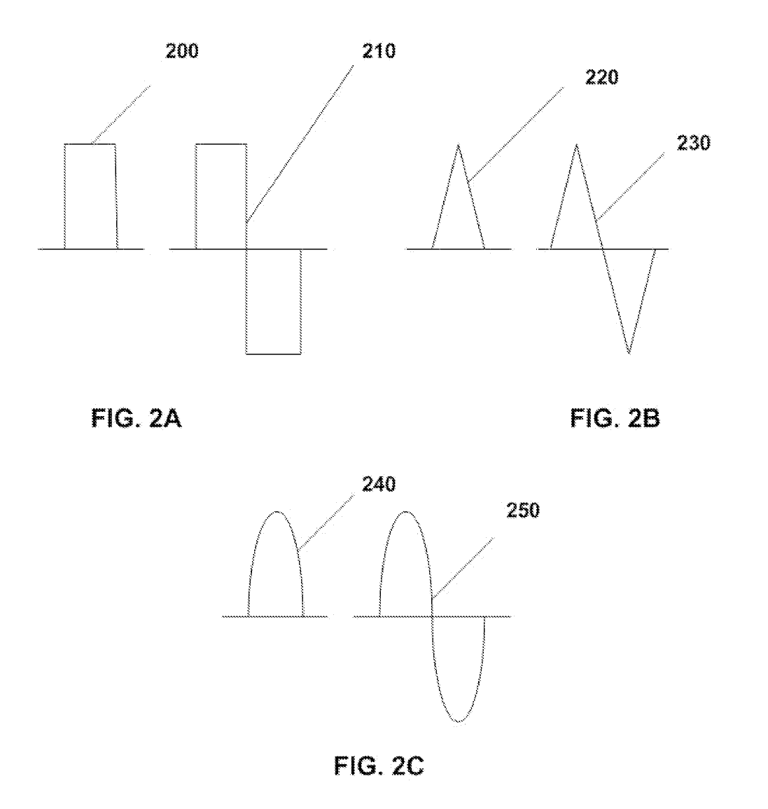

FIG. 2 shows the micro-pulse shapes that can make up the macro-pulse shapes. FIG. 2A illustrates monophasic square-wave pulse 200 and biphasic square-wave pulse 210. FIG. 2B illustrates monophasic triangular pulse 220 and biphasic triangular pulse 230. FIG. 2C illustrates monophasic sinusoidal pulse 240 and biphasic sinusoidal pulse 250.

Other embodiments can be used with different shapes including those created by signal generators capable of producing arbitrary shapes. The pulse shape can affect the effectiveness of the stimulation and that may vary by ultrasound target. Pulse lengths can be with initial rise times on the 100 microseconds with total pulse length of hundreds of microseconds to one millisecond or more. Another facet of the stimulation is the shape of the pulse and whether the pulse is monophasic or biphasic. As to repetition rate, rates on the order of 1 Hz or less typically down-regulate and several Hz. and above up-regulate.

Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results. Alternatively, the effectiveness of macro-pulse or micro-pulse neuromodulation can be judged by stimulation the palm and assessing the impact of finger movements.

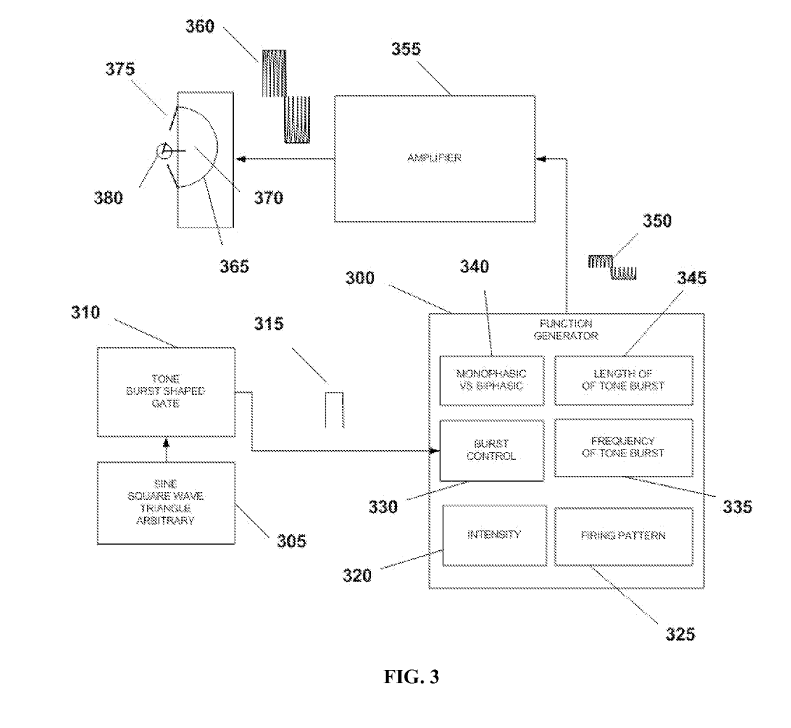

The system for generating the macro- and micro-pulse shapes is shown in FIG. 3. The macro-pulse shape (in this case a square wave) is generated by tone-burst-shaped gate 310 driven by shape control (sine, square-wave, triangle, or arbitrary) 305. The output of tone-burst-shaped gate 310 is 315 and provides input to burst control 330 of function generator 300. The other elements controlled are frequency-of-tone-burst control 335, intensity control 320, firing-pattern control 325, monophasic versus biphasic control 340, length-of-tone-burst control 345. The ultrasound transducer is pulsed with tone burst durations of (but not limited to) 25 to 500 μsec. The resulting output (in this case square-wave macro-pulse made up of sine-wave micro-pulses) 350 provides input to amplifier (for example AB linear) 355 that provides the increased power as output, shown as increased amplitude pulses 360. This drives ultrasound transducer 365 with ultrasound conduction medium 370 generating focused ultrasound field 375 aimed at neural target 380. For any ultrasound transducer position, ultrasound transmission medium (e.g., Dermasol from California Medical Innovations or silicone oil in a containment pouch) and/or an ultrasonic gel layer. Depending on the focal length of the ultrasound field, the length of the ultrasound transducer assembly can be increased with a corresponding increase in the length of ultrasound-conduction-medium insert. The focus of ultrasound transducer 365 can be purely through the physical configuration of its transducer array (e.g., the radius of the array) with an optional lens or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well.

Transducer arrays of the type 365 may also be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” ^{2nd} International Symposium on Therapeutic Ultrasound—Seattle−31/07—Feb. 8, 2002), typically with numbers of ultrasound transducers of 300 or more. The design of the individual array elements and power applied will determine whether the ultrasound is high intensity or low intensity (or medium intensity) and because the ultrasound transducers are custom, any mechanical or electrical changes can be made, if and as required.

In another embodiment the pulses (macro-shaped; micro-shaping is not applicable) of Transcranial Magnetic Stimulation (TMS) are shaped.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A system of non-invasively stimulating neural structures such as the brain using ultrasound stimulation, the system comprising:
aiming an ultrasound transducer at the selected neural target,
macro-shaping the pulse outline of the tone burst,
applying pulsed power to said ultrasound transducer via a control circuit thereby
whereby the neural structure is neuromodulated

2. The system of claim 1, wherein the macro-pulse shape is selected from the group consisting of sine wave, square wave, triangular wave, and arbitrary wave.

3. The system of claim 1, wherein the macro pulses are selected from the group consisting of homogeneous and heterogeneous.

4. The system of claim 1, wherein the macro-pulse shape is made up of micro-pulse shapes selected from the group consisting of sine wave, square wave, triangular wave, and arbitrary wave.

5. The system of claim 4, wherein the micro pulses are selected from the group consisting of homogeneous and heterogeneous.

6. The system of claim 1, wherein the plurality of control elements is selected from the group consisting of intensity, frequency, pulse duration, and firing pattern.

7. The system of claim 1, further comprising focusing the sound field of an ultrasound transducer at the target nerves neuromodulating the activity of the target in a manner selected from the group of up-regulation and down-regulation.

8. The system of claim 1, wherein the configuration of ultrasound power is selected from the group consisting of monophasic and biphasic.

9. The system of claim 1, wherein the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

10. The system of claim 1, wherein the neuromodulation results in a durable effect selected from the group consisting of Long-Term Potentiation and Long-Term Depression.

11. The system of claim 1, wherein the disorder treated is selected from the group consisting of addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

12. The system of claim 1, wherein the disorder treated is applied to the group consisting of cognitive enhancement, hedonic stimulation, enhancement of neural plasticity, improvement in wakefulness, brain mapping, diagnostic applications, and research functions.

13. The system of claim 1, wherein the invention is applied to globally depress neural activity as in the early treatment of head trauma or other insults to the brain.

14. The system of claim 1, wherein the efficacy of the macro-pulse neuromodulation is judged via an imaging mechanism selected from the group consisting of fMRI, Positron Emission Tomography, and other.

15. The system of claim 1, wherein the efficacy of the micro-pulse neuromodulation is judged via an imaging mechanism selected from the group consisting of fMRI, Positron Emission Tomography, and other.

16. The system of claim 1, wherein the effectiveness of macro-pulse neuromodulation is judged via stimulating motor cortex and assessing the magnitude of motor evoked potentials.

17. The system of claim 1, wherein the effectiveness of micro-pulse neuromodulation is judged via stimulating motor cortex and assessing the magnitude of motor evoked potentials.

18. The system of claim 1, wherein the effectiveness of macro-pulse neuromodulation is judged by stimulation the palm and assessing the impact of finger movements.

19. The system of claim 1, wherein the effectiveness of micro-pulse neuromodulation is judged by stimulation the palm and assessing the impact of finger movements.

20. The system of claim 1, wherein the Transcranial Magnetic Stimulation pulses rather than ultrasound pulses are shaped
